(19) United States
(12) Patent Application Publication
(10) Pub. No.: US 2009/0088423 A1
(43) Pub. Date: Apr. 2, 2009

(54) METHODS AND COMPOSITIONS FOR TREATING BACTERIAL INFECTIONS AND DISEASES ASSOCIATED THEREWITH

(75) Inventor: Chalom B. Sayada, Luxembourg City (LU)

Correspondence Address: INTELLECTUAL PROPERTY / TECHNOLOGY LAW, PO BOX 14329, RESEARCH TRIANGLE PARK, NC, 27709, US

(73) Assignee: ACTIVBIOTICS PHARMA LLC, Tucker, GA (US)

(21) Appl. No.: 12/255,189

(22) Filed: Oct. 21, 2008

Related U.S. Application Data
(63) Continuation of application No. 10/651,865, filed on Aug. 29, 2003, now abandoned.
Application No. 10/651,865 is a continuation-in-part of application No. 10/443,351, filed on May 22, 2003.
(60) Provisional application No. 60/382,805, filed on May 23, 2002.
(60) Provisional application No. 60/444,570, filed on Feb. 3, 2003.

(51) Int. Cl. A61K 31/5383; A61P 31/04

(52) U.S. Cl. 514/229.5

(57) ABSTRACT

The invention features methods and compositions for treating bacterial infections.

### CROSS-REFERENCE TO RELATED APPLICATIONS

This application is a continuation-in-part of U.S. Utility application Ser. No. 10/443,351, filed May 22, 2003, which claims the benefit of U.S. Provisional Application No. 60/382,805, filed May 23, 2002. This application also claims the benefit of U.S. Provisional Application No. 60/444,570, filed Feb. 3, 2003. Each of above applications is hereby incorporated by reference.

### BACKGROUND OF THE INVENTION

This invention relates to the field of treatment of bacterial infections.

Bacteria have two general growth states, a multiplying phase and a non-multiplying phase. To date, most antibiotics have been developed against bacteria in the multiplying phase (i.e., multiplying bacteria). The non-multiplying form is highly resistant to most known antibiotics. This resistance is reversible; when non-multiplying bacteria start to multiply, they become sensitive to antibiotics.

In treating a bacterial infection, the multiplying bacteria are killed by antibiotics, whereas non-multiplying or slowly multiplying bacteria tolerate repeated doses of antibiotics, leading to the need for a longer course of treatment. If the antibiotic treatment is stopped before the pool of non-multiplying bacteria has been substantially reduced or eliminated, clinical relapse is likely to occur.

One drawback to prolonged treatment is the emergence of resistance. The emergence of resistance to antibacterial agents is a pressing concern for human health. In the last decade, the frequency and spectrum of antimicrobial-resistant infections has increased. Certain infections that are essentially untreatable are reaching epidemic proportions in both the developing world and institutional settings in the developed world. Antimicrobial resistance is manifested in increased morbidity, mortality, and health-care costs. Staphylococcus aureus is a significant cause of nosocomial and community acquired infections, including skin and soft tissue infection, surgical wound infection, nosocomial pneumonia, and bloodstream infection (see, for example, Panlilio et al., Infect. Cont. Hosp. Epidemiol. 13: 582-586, 1992). Other pathogens commonly associated with serious infections include, but are not limited to, Staphylococcus spp., Streptococcus spp., Enterococcus spp., and Enterobacter spp. A considerable amount of effort has been devoted to developing antibacterial (bacteriostatic and/or bactericidal) agents with activity against these and other microorganisms.

Resistant bacteria are often present in healthy human commensal bacterial flora. Prolonged suboptimal bactericidal concentrations can lead to the emergence of resistant forms of the normal flora in the gut, skin, and throat. Non-multiplying bacteria will tend to survive standard antimicrobial therapy, and may even have an enhanced ability to mutate (see, e.g., Martinez et al., Antimicrob. Agents Chemother. 44:1771-1777, 2000; Riesenfeld et al., Antimicrob. Agents Chemother. 41:2059-2060, 1997; Alonso et al., Microbiology 145:2857-2862, 1999).

Thus there is a need for identifying therapies capable of reducing the number of non-multiplying bacteria as well as the number of multiplying bacteria, in order to provide alternative and improved methods for the treatment of bacterial infections.

### SUMMARY OF THE INVENTION

We have discovered that rifamycin antibiotics of formula (I) are effective against non-multiplying bacteria. In view of this discovery, any of these rifamycins can be employed in conjunction with antibiotics that are effective against multiplying bacteria to treat any of a wide variety of bacterial infections and associated diseases. A rifamycin antibiotic of formula (I) may be administered after treatment with such an antibiotic has been completed. Alternatively, the compound may be administered during all or part of the period during which the antibacteria effective against multiplying bacteria is being administered.

Accordingly, the invention features a method for treating a patient diagnosed as being infected with a bacterium by administering to the patient (i) a rifamycin antibiotic of formula (I), shown below, and (ii) a second antibiotic that is effective against the multiplying form of the bacterium, wherein the two antibiotics are each administered in an amount and for a duration that together treat the patient.

The invention also features a method for treating a patient diagnosed as being infected with a bacterium by administering to the patient a rifamycin antibiotic of formula (I) and a second antibiotic, wherein the two antibiotics are each administered in an amount and for a duration that together treat the patient.

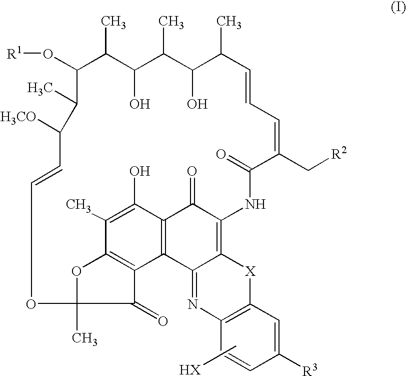

In formula (I), X represents O, S, or NR⁸, R¹ represents a hydrogen or an acetyl group, R² represents a hydrogen or hydroxyl group, and R³ represents a group expressed by the formula:

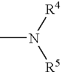

wherein each of R⁴ and R⁵ is, independently, an alkyl group having 1 to 7 carbon atoms, or R⁴ and R⁵ combine to form a 3-8 membered cyclic system,

or R³ represents a group expressed by the formula:

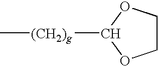

in which g represents an integer between 1 and 3;

or R³ represents a group expressed by the formula:

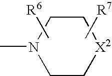

wherein each of R⁶ and R⁷ is, independently, a hydrogen atom or an alkyl group having 1 to 3 carbon atoms, X² represents an oxygen atom, a sulfur atom, or a carbonyl group,

or X² represents a group expressed by the formula:

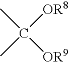

in which each of R⁸ and R⁹ is, independently, a hydrogen atom, or an alkyl group having 1 to 3 carbon atoms, or R⁸ and R⁹, in combination with each other, represent —(CH₂)ₖ— in which k represents an integer between 1 and 4;

or X² represents a group expressed by the formula:

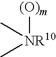

in which m represents 0 or 1, R¹⁰ represents a hydrogen atom, an alkyl group having 1 to 6 carbon atoms, or —(CH₂)ₙX³ in which n represents an integer between 1 and 4, and X³ represents an alkoxy group having 1 to 3 carbon atoms, a vinyl group, an ethynyl group,

or X2 represents a group expressed by the formula:

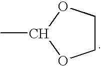

The foregoing formula describes a family of rifamycin antibiotics. Particular rifamycin antibiotics that fit this formula are disclosed in U.S. Pat. Nos. 4,690,919; 4,983,602; 5,786,349; 5,981,522; 6,316,433 and 4,859,661, each of which is hereby incorporated by reference. In a preferred embodiment of the first aspect, the rifamycin antibiotic is described by formula (II).

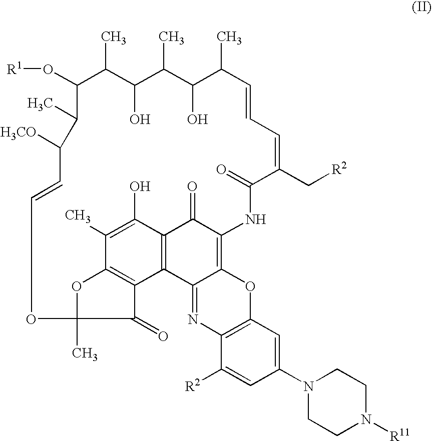

In formula (II), R represents a hydrogen or a hydroxyl group; R¹ represents hydrogen or an acetyl group; R² is hydroxyl or sulfhydryl; and R¹¹ is selected from the group consisting of methyl, ethyl, iso-propyl, n-propyl, iso-butyl, (S)-sec-butyl, and (R)-sec-butyl.

One particularly preferred rifamycin antibiotic is rifalazil. The daily dosage of rifalazil can range from 0.01 mg to 1000 mg. The daily dosage of rifalazil is normally about 1 to 1000 mg (desirably about 1 to 100 mg, more desirably about 1 to 50 mg, and even more desirably about 1 to 25 mg). The rifalazil may be given daily (e.g., once, twice, three times, or four times daily) or less frequently (e.g., once every other day, once or twice weekly, or monthly). Treatment may be for 1 day to 1 year, or longer. Desirably, treatment is for 1 to 21 days, and more desirably for 1 to 14 days or even 1, 3, 5, or 7 days. In another embodiment, rifalazil is administered at an initial dose of between 5 and 100 mg, followed by subsequent doses of between 1 and 50 mg for 3 to 7 days. A single dose of rifalazil (e.g., in a dosage of between 1 and 100 mg) may also be employed in a method of the invention. The rifalazil may be administered orally, intravenously, subcutaneously, or rectally in a method of the invention.

In one embodiment of the invention, the method includes administering rifalazil and vancomycin simultaneously or sequentially. Rifalazil and vancomycin can be administered within fourteen days of each other, preferably within five days, more preferably within three days and most preferably within twenty-four hours of each other. If desired, either rifalazil or vancomycin, or both can be administered orally. Dosages for vancomycin can range from 20 to 2000 mg per day or higher (e.g., 4000 mg or the maximal tolerated dosage), preferably from 125 to 2000 mg per day, most preferably from 500 to 2000 mg per day.

The patient can be any warm-blooded animal including but not limited to a human, cow, horse, pig, sheep, bird, mouse, rat, dog, cat, monkey, baboon, or the like. It is most preferred that the patient be a human.

In one preferred method of carrying out the foregoing method, the antibiotic that is effective against the multiplying form of the bacterium (e.g., vancomycin) is administered in an amount and for a duration to reduce the number of bacteria in the patient to less than about 10⁶ organisms/mL. This typically takes from a few hours to 1, 2, or 3 days, but may take as long as a week. After this has been achieved, the patient is then administered a rifamycin antibiotic of formula (I) or formula (II) (e.g., rifalazil) in an amount and for a duration sufficient to complete the treatment of the patient.

In another preferred method, the rifamycin of formula (I) is administered in an amount and for a duration that, in combination with the second antibiotic, decreases the patient's bacterial load by at least one, two, or three orders of magnitude within 24, 48, or 72 hours.

If desirable, the administration of the first antibiotic can be continued while the rifamycin antibiotic is being administered.

In one particularly desirable embodiment, the rifamycin antibiotic is administered orally or intravenously, while the antibiotic effective against multiplying bacteria is administered intravenously.

The methods of the present invention can be used to treat, for example, respiratory tract infections (e.g., inhalation anthrax), acute bacterial otitis media, bacterial pneumonia, urinary tract infections, complicated infections, noncomplicated infections, pyelonephritis, intra-abdominal infections, deep-seated abcesses, bacterial sepsis, skin and skin structure infections (e.g., cutaneous anthrax), soft tissue infections (e.g., endometritis), bone and joint infections (e.g., osteomyelitis, septic arthritis), central nervous system infections (e.g., meningitis), bacteremia, wound infections, peritonitis, meningitis, infections after burn, urogenital tract infections, gastro-intestinal tract infections (e.g., antibiotic-associated colitis, gastrointestinal anthrax), pelvic inflammatory disease, and endocarditis.

The methods of the present invention can also be used to treat diseases associated with bacterial infection. For example, bacterial infections can produce inflammation, resulting in the pathogenesis of atherosclerosis, multiple sclerosis, rheumatoid arthritis, diabetes, Alzheimer's disease, asthma, cirrhosis of the liver, psoriasis, meningitis, cystic fibrosis, cancer, or osteoporosis. Accordingly, the present invention also features a method of treating the diseases associated with bacterial infection listed above.

The methods of the present invention can be used to treat or prevent infections by bacteria from a variety of genera, such as Escherichia spp., Enterobacter spp., Enterobacteriaceae spp., Klebsiella spp., Serratia spp., Pseudomonas spp., Acinetobacter spp., Bacillus spp., Micrococcus spp., Arthrobacter spp., Peptostreptococcus spp., Staphylococcus spp., Enterococcus spp., Streptococcus spp., Haemophilus spp., Neisseria spp., Bacteroides spp., Citrobacter spp., Branhamella spp., Salmonella spp., Shigella spp., Proteus spp., Clostridium spp., Erysipelothrix spp., Listeria spp., Pasteurella spp., Streptobacillus spp., Spirillum spp., Fusospirocheta spp., Treponema spp., Borrelia spp., Actinomycetes spp., Mycoplasma spp., Chlamydia spp., Rickettsia spp., Spirochaeta spp., Legionella spp., Mycobacteria spp., Ureaplasma spp., Streptomyces spp., and Trichomoras spp. Accordingly, the invention features a method of treating infections by the bacteria belonging to the genera above, among others.

Particular Gram-positive bacterial infections that can be treated according to the method of the invention are infections by Staphylococcus aureus, Staphylococcus epidermidis, Enterococcus faecalis, Enterococcus faecium, Streptococcus pyogenes, Streptococcus pneumoniae, Streptococcus mutans, Streptococcus agalactiae, Bacillus anthracis, Bacillus cereus, Clostridium perfringens, Clostridium tetani, Clostridium botulinum, and Clostridium difficile.

Multi-drug resistant strains of bacteria can be treated according to the methods of the invention. Resistant strains of bacteria include penicillin-resistant, methicillin-resistant, quinolone-resistant, macrolide-resistant, and/or vancomycin-resistant bacterial strains. Multi-drug resistant bacterial infections to be treated using the methods of the present invention include infections by penicillin-, methicillin-, macrolide-, vancomycin-, and/or quinolone-resistant Streptococcus pneumoniae; penicillin-, methicillin-, macrolide-, vancomycin-, and/or quinolone-resistant Staphylococcus aureus; penicillin-, methicillin-, macrolide-, vancomycin-, and/or quinolone-resistant Streptococcus pyogenes; and penicillin-, methicillin-, macrolide-, vancomycin-, and/or quinolone-resistant enterococci.

The invention also features a method of eradicating non-multiplying bacteria not eradicated in a patient following treatment with a first antibiotic by administering to the patient a rifamycin antibiotic of formula (I) or (II) in an amount and for a duration sufficient to eradicate the non-multiplying bacteria in the patient.

In another aspect, the invention features a method of treating a patient diagnosed as having a chronic disease associated with a bacterial infection caused by bacteria capable of establishing a cryptic phase. The method includes the step of administering to a patient a rifamycin antibiotic of formula (I) or (II).

In yet another aspect, the invention features a method of treating the cryptic phase of a bacterial infection. This method includes the step of administering to a patient a rifamycin of formula (I) or (II) or any of the preferred embodiments of these formulas described above. The administering is for a time and in an amount sufficient to treat the cryptic phase of the bacterial infection.

The invention also features a method of treating a bacterial infection in a patient by (a) treating the multiplying form of the bacteria by administering an antibiotic to the patient for a time and an amount sufficient to treat the multiplying form, and (b) treating the non-multiplying form of the bacteria by administering to the patient a rifamycin antibiotic of formula (I) or (II), wherein the administering is for a time and in an amount sufficient to treat the non-multiplying form.

The time sufficient to treat a non-multiplying form of a bacterium ranges from one day to one year. In certain instances, a single oral dose of a rifamycin antibiotic of formula (I) may be sufficient to treat an infection having a cryptic phase or other non-multiplying form. Treatment can also be for several weeks or months, or even extended over the lifetime of the individual patient, if necessary. For example, the duration of treatment may be at least 30 days, at least 45 days, at least 90 days, or at least 180 days. Ultimately, it is most desirable to extend the treatment for such a time that the non-multiplying form is no longer detectable.

The invention also features a pharmaceutical composition that includes (i) a rifamycin antibiotic of formula (I) and a second antibiotic selected from penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minoc ycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, and trimethoprim.

In any of the methods and compositions of the invention, one or more additional antibiotics (e.g., any of the antibiotics listed above) may be employed in addition to the rifamycin of formula (I) and the second antibiotic.

For the purpose of the present invention, the following abbreviations and terms are defined below.

By “alkoxy” is meant a chemical substituent of the formula —OR, wherein R is an alkyl group.

By “alkyl” is meant a branched or unbranched saturated hydrocarbon group, desirably having from 1 to 10 carbon atoms. An alkyl may optionally include monocyclic, bicyclic, or tricyclic rings, in which each ring desirably has three to six members. The alkyl group may be substituted or unsubstituted. Exemplary substituents include alkoxy, aryloxy, sulfhydryl, alkylthio, arylthio, halogen, hydroxy, fluoroalkyl, perfluoralkyl, amino, aminoalkyl, disubstituted amino, quaternary amino, hydroxyalkyl, carboxyalkyl, and carboxyl groups.

In various embodiments of the invention the alkyl group is of 1 to 10 carbon atoms. Exemplary substituents include methyl; ethyl; n-propyl; isopropyl; n-butyl; iso-butyl; sec-butyl; tert-butyl; pentyl; cyclopropyl; cyclobutyl; cyclopentyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-l-methylpropyl; 1-ethyl-2-methylpropyl; hexyl; heptyl; cyclohexyl; cycloheptyl; and cyclooctyl.

By “administering” is meant a method of giving one or more unit doses of an antibacterial pharmaceutical composition to an animal (e.g., topical, oral, intravenous, intraperitoneal, or intramuscular administration). The method of administration may vary depending on various factors, e.g., the components of the pharmaceutical composition, site of the potential or actual bacterial infection, bacteria involved, and severity of the actual bacterial infection.

By “an amount effective to treat” is meant the amount of a drug required to treat or prevent an infection or a disease associated with an infection. The effective amount of a drug used to practice the present invention for therapeutic or prophylactic treatment of conditions caused by or contributed to by a microbial infection varies depending upon the manner of administration, the age, body weight, and general health of the subject. Ultimately, the attending physician will decide the appropriate amount and dosage regimen. Such amount is referred to as an “effective” amount.

By “bacteria” is meant a unicellular prokaryotic microorganism that usually multiplies by cell division.

By “bacteria capable of establishing a cryptic phase” is meant any species whose life cycle includes a persistent, non-multiplying phase. These species include but are not limited to C. trachomatis, C. pneumoniae, C. psittaci, C. suis, C. pecorum, C. abortus, C. caviae, C. felis, C. muridarum, N. hartmannellae, W. chondrophila, S. negevensis, and P. acanthamoeba, as well as any other species described in Everett et al. (Int. J. Syst. Evol. Microbiol. 49:415-440, 1999).

By “bacterial infection” is meant the invasion of a host animal by pathogenic bacteria. For example, the infection may include the excessive growth of bacteria that are normally present in or on the body of an animal or growth of bacteria that are not normally present in or on the animal. More generally, a bacterial infection can be any situation in which the presence of a bacterial population(s) is damaging to a host animal. Thus, an animal is “suffering” from a bacterial infection when an excessive amount of a bacterial population is present in or on the animal's body, or when the presence of a bacterial population(s) is damaging the cells or other tissue of the animal.

By “cryptic phase” is meant the latent or dormant intracellular phase of infection characterized by little or no metabolic activity. The non-multiplying cryptic phase is often characteristic of persistent forms of intracellular bacterial infections.

By “elementary body phase” is meant the infectious phase of the bacterial life cycle which is characterized by the presence of elementary bodies (EBs). EBs are small (300-400 nm), infectious, spore-like forms which are metabolically inactive, non-multiplying, and found most often in the acellular milieu. EBs possess a rigid outer membrane which protects them from a variety of physical insults such as enzymatic degradation, sonication and osmotic pressure.

By “intracytoplasmic inclusion” is meant a multiplying reticulate body (RB) that has no cell wall. Such inclusions may be detected, for example, through chlamydiae sample isolation and propagation on a mammalian cell lines, followed by fixing and staining using one of a variety of staining methods including Giemsa staining, iodine staining, and immunofluorescence. These inclusions have a typical round or oval appearance.

By “persistent bacterial infection” is meant an infection that is not completely eradicated through standard treatment regimens using antibiotics. Persistent bacterial infections are caused by bacteria capable of establishing a cryptic phase or other non-multiplying form of a bacterium and may be classified as such by culturing bacteria from a patient and demonstrating bacterial survival in vitro in the presence of antibiotics or by determination of anti-bacterial treatment failure in a patient. As used herein, a persistent infection in a patient includes any recurrence of an infection, after receiving antibiotic treatment, from the same species more than two times over the period of two or more years or the detection of the cryptic phase of the infection in the patient. An in vivo persistent infection can be identified through the use of a reverse transcriptase polymerase chain reaction (RT-PCR) to demonstrate the presence of 16S rRNA transcripts in bacterially infected cells after treatment with one or more antibiotics (Antimicrob. Agents Chemother. 12:3288-3297, 2000).

By “autoimmune disease” is meant a disease arising from an immune reaction against self-antigens and directed against the individual's own tissues. Examples of autoimmune diseases include but are not limited to systemic lupus erythematosus, rheumatoid arthritis, myasthenia gravis, and Graves' disease.

By “chronic disease” is meant a disease that is inveterate, of long continuance, or progresses slowly, in contrast to an acute disease, which rapidly terminates. A chronic disease may begin with a rapid onset or in a slow, insidious manner but it tends to persist for several weeks, months or years, and has a vague and indefinite termination.

By “immunocompromised” is meant a person who exhibits an attenuated or reduced ability to mount a normal cellular or humoral defense to challenge by infectious agents, e.g., viruses, bacterial, fungi, and protozoa. Persons considered immunocompromised include malnourished patients, patients undergoing surgery and bone narrow transplants, patients undergoing chemotherapy or radiotherapy, neutropenic patients, HIV-infected patients, trauma patients, bum patients, patients with chronic or resistant infections such as those resulting from myelodysplastic syndrome, and the elderly, all of who may have weakened immune systems.

By “inflammatory disease” is meant a disease state characterized by (1) alterations in vascular caliber that lead to an increase in blood flow, (2) structural changes in the microvasculature that permit the plasma proteins and leukocytes to leave the circulation, and (3) emigration of the leukocytes from the microcirculation and their accumulation in the focus of injury. The classic signs of acute inflammation are erythema, edema, tenderness (hyperalgesia), and pain. Chronic inflammatory diseases are characterized by infiltration with mononuclear cells (e.g., macrophages, lymphocytes, and plasma cells), tissue destruction, and fibrosis. Non-limiting examples of inflammatory disease include asthma, coronary artery disease, arthritis, conjunctivitis, lymphogranuloma venerum, and salpingitis.

By “treating” is meant administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve the patient's condition. Thus, in the claims and embodiments, treating is the administration to a mammal either for therapeutic or prophylactic purposes.

The present invention satisfies an existing need for antibiotics that are effective in the treatment of bacterial infections caused by bacteria capable of establishing a non-multiplying phase of infection, or diseases associated with these bacterial infections. The invention described herein allows for a more complete treatment of a bacterial infection by targeting both the multiplying and non-multiplying phase of the bacteria responsible for the infection. The treatment methods of the invention may improve compliance, reduce the emergence of resistance, and shorten the course of treatment.

Other features and advantages of the invention will be apparent from the following description of the preferred embodiments thereof, and from the claims.

### BRIEF DESCRIPTION OF THE DRAWINGS

FIG. 1 is a graph showing cfu/ml from S. aureus 29213 cultures exposed to rifampicin (Rif) or rifalazil (Rfz) alone (at 0.1 μg/ml) or in combination with vancomycin (Van; 10 μg/ml).

FIG. 2 is a graph showing the number of Rif-resistant cfu/ml present in S. aureus cultures exposed to Rif and Rfz, alone or in combination with Van (10 μg/ml).

FIG. 3 is a graph showing cfu/ml of S. aureus 29213 in cultures exposed to Rfz alone (0.1 μg/ml or 0.025 μg/ml) or in combination with Van (15 μg/ml).

FIG. 4 is a graph showing Rif-resistant cfu/ml present in S. aureus cultures exposed to Rfz (0.1 or 0.025 μg/ml) alone or in combination with Van (15 μg/ml).

FIG. 5 is a graph showing the effect of Rfz and Van alone or in combination on stationary-phase cells of S. aureus 29213.

FIG. 6 is a graph showing Rif-resistant cfu/ml in stationary phase Rfz/Van-treated S. aureus.

### DETAILED DESCRIPTION OF THE INVENTION

We have discovered that the rifamycin antibiotics of formula (I) are effective against non-multiplying bacteria, and that the use of such antibiotics in conjunction with antibiotics that are effective against the multiplying form of the same bacteria results in shorter, more effective treatment of an infected patient, reduces the opportunity for the emergence of antibiotic resistance, and allows for the earlier discharge of the patient from a hospital.

One exemplary combination of antibiotics is rifalazil (Rfz) and vancomycin (Van). As is described in detail below, we characterized the antibacterial effect of Rfz on both multiplying logarithmic (log)-phase and non-multiplying stationary-phase cultures of Staphylococcus aureus ATCC 29213 (“wild-type” S. aureus). The combination of Rfz with vancomycin (Van) was also tested. Cultures were grown in flasks to about 5×10⁸ colony-forming units (cfu) per ml for log-phase and 5×10⁹ cfu per ml for stationary-phase, and then treated with single drugs or the combination thereof. The viability of the cultures was monitored by plating aliquots on non-selective plates. The presence in the cultures of Rfz-resistant mutants was assessed by plating aliquots on Rif-containing plates. (Rfz-resistant mutants are cross-resistant to Rif, so for convenience, agar plates containing Rif were utilized for tittering Rfz-resistant mutants.) When used as a single agent for log-phase cultures, Rfz was found to select for Rif-resistant mutants, as evidenced by a dramatic increase in Rif-resistant cfu/ml within 10 hours. Co-treatment with Van delayed or arrested the appearance of Rif-resistant cfu in log-phase cultures. Rfz and Van used singly were found to be less effective at killing stationary-phase cultures (approximately 1 log killing over 48 hours). Rfz in combination with Van was able to kill stationary phase cultures (3 to 4 log killing over 48 hours). These studies suggest that combination of Rfz and Van may have utility in in vivo infection models and possibly in the clinic.

The experiments described below were carried out to assess the antibacterial activity of Rfz, alone or in combination with Van on multiplying and non-multiplying forms of S. aureus ATCC 29213. In log-phase, Rfz treatment results in an initial rapid bactericidal effect, followed by a recovery of cfu/ml over a 6-48 hour period. The recovery represents an outgrowth of Rfz -resistant mutant cells that were present in the culture as a sub-population at the start of the experiment. When combined with Van, the emergence of Rfz-resistant mutants in cultures was very significantly delayed or arrested. It is assumed that the Van present in these cultures is responsible for inhibiting the growth, or killing, of part of the Rif/Rfz-resistant mutant subpopulation present at the start of the experiments. Van was slowly bactericidal in these experiments, that is, significant killing (3 logs) only occurred after 48 hours of treatment. In addition, Van was subject to a concentration-dependent effectiveness with the dense cultures used in these experiments; 15×MIC levels of the drug had to be used to get a bactericidal effect as compared to 6.5×MIC levels for Rfz for these dense cultures. In fact, it was found that 1.6× the MIC of Rfz, in combination with 15 μg/ml vancomycin, or alone, was as effective as 6.5x the MIC levels of Rfz. The combination of Rfz and Van provides a cidality that is more rapid than Van used alone and may avoid the issue of resistance development associated with Rfz used as a single agent.

Rfz and Van had little or no killing effect on their own against stationary phase cultures, while the combination of Rfz and Van demonstrated significant reduction of cfu/ml in stationary phase (non-multiplying) cultures. Because such a population or subpopulation of stationary-phase cells might be expected to exist in an in vivo infection, or infection model, it appears that a Rfz/Van combination might prove efficacious as compared to each of the drugs being used alone.

### Rifamycin Antibiotics

Rifamycins are a group of antibiotics that belong to a class of antibiotics called ansamycins. The rifamycin antibiotics that can be employed in the present invention are disclosed in U.S. Pat. Nos. 4,690,919; 4,983,602; 5,786,349; 5,981,522; 6,316,433 and 4,859,661 each of which is hereby incorporated by reference. In preferred embodiments, the rifamycin antibiotic employed in the methods and compositions of the present invention is Rfz, ABI1657, or ABI 131. The specific chemical formula of Rfz is that of formula II wherein R is a hydrogen atom; R¹ is an acetyl group; R² is a hydroxyl group; and R¹¹ is an iso-butyl group. The specific chemical formula of KRM 1657 is that of formula II wherein R is a hydrogen atom; R¹ is an acetyl group; R² is a hydroxyl group; and R¹¹ is an n-propyl group. The specific chemical formula of KRM1131 is that of formula II wherein R is a hydrogen atom; R¹ is an acetyl group; R² is a hydroxyl group; and R¹¹ is a methyl group.

### Antibiotics Effective Against Multiplying Bacteria

Rifamycin antibiotics of formula (I) can be administered before, during, or after administration of another or more than one antibiotic; in the methods of the invention, these other antibiotics are effective against multiplying bacteria. Exemplary antibiotics that are effective against multiplying bacteria and thus can be administered in the methods of the invention are β-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenams (e.g., imipenem, ertapenem, and meropenem), and monobactams (e.g., astreonam); β-lactamase inhibitors (e.g., clavulanate, sulbactam, and tazobactam); aminoglycosides (e.g., streptomycin, neomycin, kanamycin, paromycin, gentamicin, tobramycin, amikacin, netilmicin, spectinomycin, sisomicin, dibekalin, and isepamicin); tetracyclines (e.g., tetracycline, chlortetracycline, demeclocycline, minocycline, oxytetracycline, methacycline, and doxycycline); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); metronidazole; daptomycin; garenoxacin; ramoplanin; faropenem; polymyxin; tigecycline, AZD2563; and trimethoprim.

These antibiotics can be used in the dose ranges currently known and used for these agents. Different concentrations may be employed depending on the clinical condition of the patient, the goal of therapy (treatment or prophylaxis), the anticipated duration, and the severity of the infection for which the drug is being administered. Additional considerations in dose selection include the type of infection, age of the patient (e.g., pediatric, adult, or geriatric), general health, and comorbidity. Determining what concentrations to employ are within the skills of the pharmacist, medicinal chemist, or medical practitioner. Typical dosages and frequencies are provided, e.g., in the Merck Manual of Diagnosis & Therapy (17th Ed. MH Beers et al., Merck & Co.).

### Therapy

The invention features methods for treating bacterial infections and diseases associated with such infections by administering antibiotic combinations, as described herein.

Therapy according to the invention may be performed alone or in conjunction with another therapy and may be provided at home, the doctor's office, a clinic, a hospital's outpatient department, or a hospital. The duration of the therapy depends on the type of disease or disorder being treated, the age and condition of the patient, the stage and type of the patient's disease, and how the patient responds to the treatment.

In combination therapy, the dosage and frequency of administration of each component of the combination can be controlled independently. For example, one compound may be administered three times per day, while the second compound may be administered once per day. The compounds may also be formulated together such that one administration delivers both compounds.

### Formulation of Pharmaceutical Compositions

Administration of a compound may be by any suitable means that is effective for the treatment of a bacterial infection or associated disease. Compounds are admixed with a suitable carrier substance, and are generally present in an amount of 1-95% by weight of the total weight of the composition. The composition may be provided in a dosage form that is suitable for oral, parenteral (e.g., intravenous, intramuscular, subcutaneous), rectal, transdermal, nasal, vaginal, inhalant, or ocular administration. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols. The pharmaceutical compositions may be formulated according to conventional pharmaceutical practice (see, e.g., Remington: The Science and Practice of Pharmacy, (20th ed.) ed. A. R. Gennaro, 2000, Lippincott Williams & Wilkins, Philadelphia, Pa. and Encyclopedia of Pharmaceutical Technology, eds. J. Swarbrick and J. C. Boylan, 1988-2002, Marcel Dekker, N.Y.). Intravenous formulations of Rfz are described in U.S. patent application Ser. No. 10/453,155 (filed Jun. 3, 2003), entitled INTRAVENOUS RIFALAZIL FORMULATION AND METHODS OF USE THEREOF.

### Bacterial Infections

The methods and compositions of the present invention can be used to treat, for example, respiratory tract infections (e.g., inhalation anthrax), acute bacterial otitis media, bacterial pneumonia, urinary tract infections, complicated infections, noncomplicated infections, pyelonephritis, intra-abdominal infections, deep-seated abcesses, bacterial sepsis, skin and skin structure infections (e.g., cutaneous anthrax), soft tissue infections (e.g., endometritis), bone and joint infections (e.g., osteomyelitis, septic arthritis), central nervous system infections (e.g., meningitis), bacteremia, wound infections, peritonitis, meningitis, infections after bum, urogenital tract infections, gastro-intestinal tract infections (e.g., antibiotic-associated colitis, gastrointestinal anthrax), pelvic inflammatory disease, and endocarditis.

Diseases Associated with Infections

Diseases associated with bacterial infections include, but are not limited to, atherosclerosis, multiple sclerosis, rheumatoid arthritis, diabetes, Alzheimer's disease, asthma, cirrhosis of the liver, psoriasis, meningitis, cystic fibrosis, cancer, and osteoporosis.

Several lines of evidence have led to the establishment of a link between bacterial infections and a broad set of inflammatory, autoimmune, and immune deficiency diseases. Thus, the present invention describes methods for treating chronic diseases associated with a persistent infection, such as autoimmune diseases, inflammatory diseases and diseases that occur in immuno-compromised individuals by treating the non-multiplying form of the infection in an individual in need thereof, by administering a rifamycin antibiotic described herein, or such a rifamycin in conjunction with an antibiotic effective against multiplying bacteria. Progress of the treatment can be evaluated, using the diagnostic tests known in the art, to determine the presence or absence of the bacteria. Physical improvement in the conditions and symptoms typically associated with the disease to be treated can also be evaluated. Based upon these evaluating factors, the physician can maintain or modify the anti-bacterial therapy accordingly.

The therapies described herein can be used for the treatment of chronic immune and autoimmune diseases when patients are demonstrated to have a bacterial infection. These diseases include, but are not limited to, chronic hepatitis, systemic lupus erythematosus, arthritis, thyroidosis, scleroderma, diabetes mellitus, Graves' disease, Beschet's disease, and graft versus host disease (graft rejection). The therapies of this invention can also be used to treat any disorders in which a bacterial infection is a factor or co-factor.

Thus, the present invention can be used to treat a range of disorders in addition to the above immune and autoimmune diseases when demonstrated to be associated with chlamydial infection by the methods of detection described herein; for example, various infections, many of which produce inflammation as primary or secondary symptoms, including, but not limited to, sepsis syndrome, cachexia, circulatory collapse and shock resulting from acute or chronic bacterial infection, acute and chronic parasitic and/or infectious diseases from bacterial, viral or fungal sources, such as a HIV, AIDS (including symptoms of cachexia, autoimmune disorders, AIDS dementia complex and infections) can be treated.

Among the various inflammatory diseases, there are certain features that are generally agreed to be characteristic of the inflammatory process. These include fenestration of the microvasculature, leakage of the elements of blood into the interstitial spaces, and migration of leukocytes into the inflamed tissue. On a macroscopic level, this is usually accompanied by the familiar clinical signs of erythema, edema, tenderness (hyperalgesia), and pain. Inflammatory diseases, such as chronic inflammatory pathologies and vascular inflammatory pathologies, including chronic inflammatory pathologies such as aneurysms, hemorrhoids, sarcoidosis, chronic inflammatory bowel disease, ulcerative colitis, and Crohn's disease and vascular inflammatory pathologies, such as, but not limited to, disseminated intravascular coagulation, atherosclerosis, and Kawasaki's pathology are also suitable for treatment by methods described herein. The invention can also be used to treat inflammatory diseases such as coronary artery disease, hypertension, stroke, asthma, chronic hepatitis, multiple sclerosis, peripheral neuropathy, chronic or recurrent sore throat, laryngitis, tracheobronchitis, chronic vascular headaches (including migraines, cluster headaches and tension headaches) and pneumonia when demonstrated to be pathogenically related to a bacterial infection.

Treatable disorders when associated with a bacterial infection also include, but are not limited to, neurodegenerative diseases, including, but not limited to, demyelinating diseases, such as multiple sclerosis and acute transverse myelitis; extrapyramidal and cerebellar disorders, such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; progressive supranucleo palsy; cerebellar and spinocerebellar disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and Machado Joseph)); and systemic disorders (Refsum's disease, abetalipoprotemia, ataxia, telangiectasia, and mitochondrial multi-system disorder); demyelinating core disorders, such as multiple sclerosis, acute transverse myelitis; disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; senile dementia of Lewy body type; Wernicke-Korsakoff syndrome; chronic alcoholism; Creutzfeldt-Jakob disease; subacute sclerosing panencephalitis, Hallerrorden-Spatz disease; and dementia pugilistica.

It is also recognized that malignant pathologies involving tumors or other malignancies, such as, but not limited to leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or mycosis fungoides)); carcinomas (such as colon carcinoma) and metastases thereof; cancer-related angiogenesis; infantile hemangiomas; and alcohol-induced hepatitis. Ocular neovascularization, psoriasis, duodenal ulcers, angiogenesis of the female reproductive tract, can also be treated when demonstrated by the diagnostic procedures described herein to be associated with a bacterial infection.

### Example 1

Treatment of Log-Phase S. aureus Cultures with Rif, Rfz and Van.

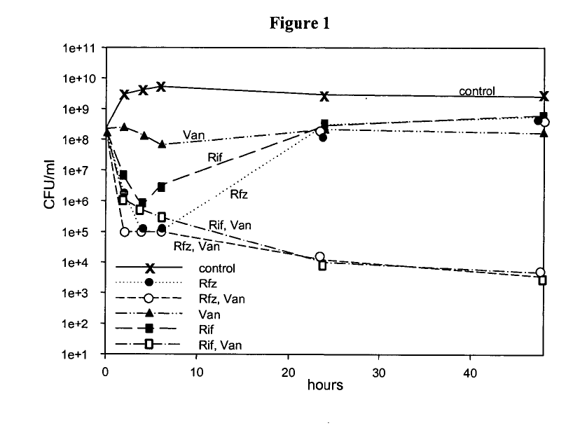

Replicate log-phase cultures of S. aureus 29213 were exposed to Rif, Rfz, Van, Rif+Van, or Rfz+Van. Viability of the cultures was monitored by plating aliquots of the cultures on non-selective MHA plates times 0, 2, 4, 6, 24 and 48 hours as described herein (FIG. 1). Rif and Rfz were used at a concentration of 0.1 μg/ml, approximately 6.5× their MIC (Rif and Rfz MIC values are each 0.015 μg/ml; these MIC values were determined according to NCCLS standard MIC testing; National Committee for Clinical Laboratory Standards. 1997. Methods for Dilution Antimicrobial Susceptibility Tests for Bacteria That Grow Aerobically-Fourth Edition: Approved Standard M7-A4. NCCLS, Villanova, Pa.). Van was used at 10 μg/ml, corresponding to 10× its MIC for the S. aureus strain.

In this experiment, Rfz alone caused a fairly rapid drop in cfu/ml, with approximately 3.5 logs killed in 4 hours. After a 24-hour period, however, the viability of the culture recovered and increased to about 1×10⁸ cfu/ml. Rif also resulted in a rapid initial drop in viability of the culture, with approximately a 2.5-log decrease in cfu/ml. Van treatment did not have a dramatic effect on the viability of the culture at 10 μg/ml, with a 0.5-log decrease in cfu/ml. Van is known to be only slowly cidal (Flandrois et al., Antimicrob. Agents Chemother. 32:454-457, 1988), but in this experiment, the killing effect of Van was not prolonged. The likely reason that Van was ineffective in this experiment when used alone is that the starting density of the culture is much higher than that which is traditionally used, thus effectively increasing the number of targets (the D-ala:D-ala portion of peptidoglycan) to be inactivated. Combined with the fact that Van is slowly cidal, this difference likely results in incomplete inhibition of the population. Additional experiments in which the vancomycin concentration was increased to 15 μg/ml, demonstrated that this higher level of vancomycin was sufficient for sustained killing. When used in combination with Van, both Rif and Rfz showed somewhat enhanced killing at the 2-hour time point, as compared to these drugs used alone. An approximately 1-log additional drop in cfu/ml was observed for the combinations. In addition, cultures treated with the combinations did not recover and cfu/ml remained at 1×10⁴ cfu/ml, even at the 48-hour time point.

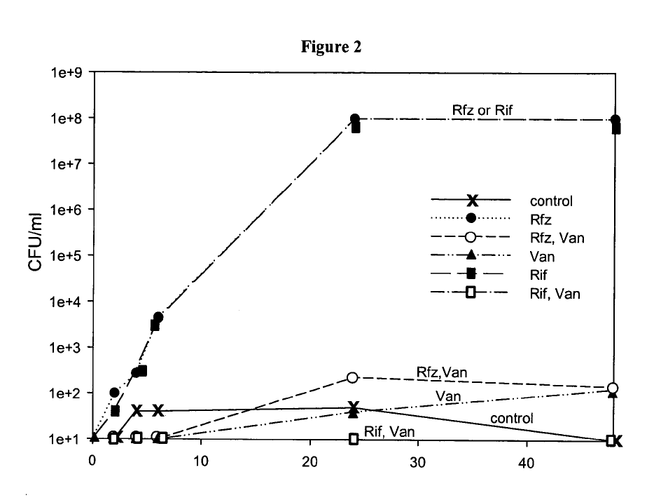

In order to characterize the population of the cultures shown in FIG. 1, aliquots at the various time points/treatments were tested for the presence of Rif-resistant mutants. Results are shown in FIG. 2. Untreated control cultures and cultures treated with Van exhibited little or no increase in the number of Rif-resistant cells. Treatment with either Rif or Rfz resulted in the rapid appearance of Rif-resistant cfu, reaching 8 logs in 24 hours. As was noted above, Rif and Rfz treatment caused an initial decrease in cfu/ml, after which the cultures resumed growth. The rise in Rif-resistant cfu reveals that the recovery of culture growth was in fact due to the emergence of Rif-resistant cells.

Emergence of Rif-resistant mutants in combination-treated cultures revealed that their outgrowth was suppressed or arrested, as compared to when Rif or Rfz were used alone (FIG. 2).

### Example 2

Treatment of Log-Phase S. aureus Cultures with Rfz Alone or in Combination with Van

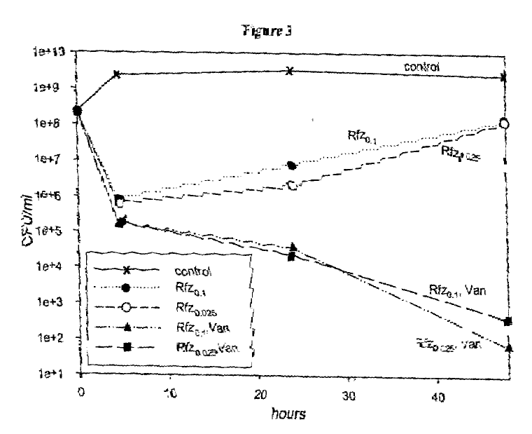

Replicate log-phase cultures of S. aureus were treated with Rfz at 0.1 μg/ml or 0.025 μg/ml alone or in combination with Van at 15 μg/ml and monitored for cfu/ml at 4.5 hours, 24 hours and 48 hours (FIG. 3). The two levels of Rfz used in this experiment are equal to 6.5× and 1.6× the MIC of Rfz. At 6.5× its MIC, Rfz was effective at reducing the cfu/ml of the culture by approximately 3 logs within 6 hours (cidality). At the lower concentration, Rfz was just as effective at reducing cfu/ml of the culture as when it was used at the higher concentration. As we observed previously, the cfu/ml levels in these cultures increased over 48 hours to approximately 1×10⁸ cfu/ml. In combination with Van, both concentrations of Rfz were able to decrease (at 4.5 hours) cfu/ml of the cultures more effectively than Rfz used alone (about ½ log greater effect). The combination-treated cultures did not exhibit extensive outgrowth by the end of the experiment.

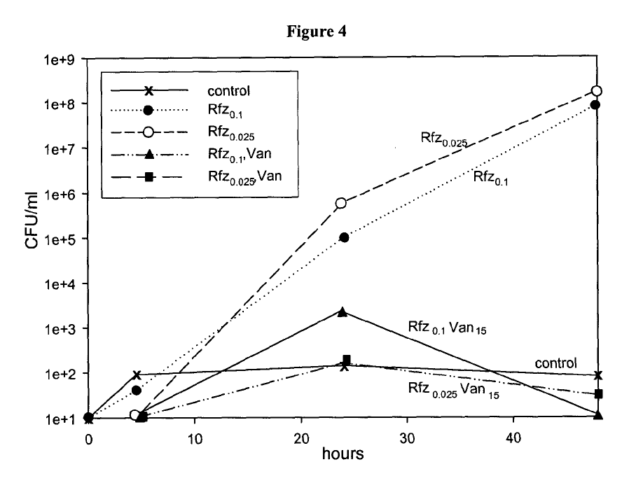

Cultures were also examined as described above for the number of cfu/ml present that were resistant to Rif (FIG. 4). Treatment with Rif at either concentration resulted in the eventual outgrowth of Rif-resistant mutant populations, as has been seen in our previous experiments. When combined with Van, the onset of resistance emergence was delayed or arrested. These data demonstrate that Rfz can be used in Van combinations at a lower concentration (1.6×MIC vs. 6.5×MIC) with similar results.

### Example 3

Treatment of Stationary-Phase S. aureus Cultures with Rfz and Van

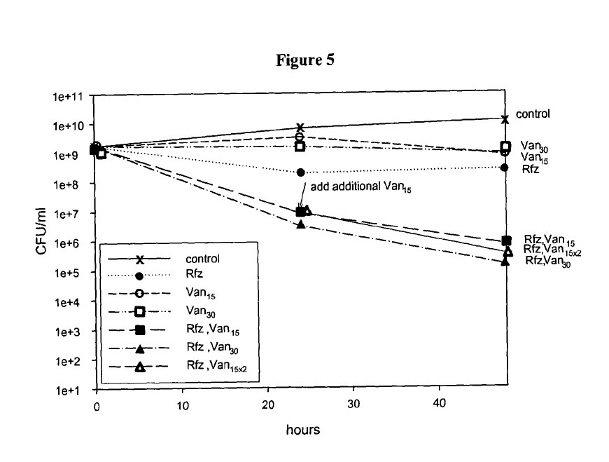

Cultures of S. aureus 29213 were grown to stationary-phase (an OD of 2.2 to 2.4 at 600 nm), then treated with Van and Rfz, either alone or in combination (FIG. 5; concentrations listed are in μg/ml). Rfz was used at 0.1 μg/ml, while Van was used at 15 μg/ml and 30 μg/ml, the lafter concentration based on the fact that the starting culture was at a much higher density than was the log-phase cultures used in the experiments described above. It was assumed that more Van might be required to give a prolonged killing effect. In addition, one culture was treated with 15 μg/ml Van (Van_{15×2}) at the start of the experiment and then again 24 hours later. At 0, 24 and 48 hours, the number of viable cells was determined by plating on non-selective medium as described above. The results are shown in FIG. 5.

The viability of the cultures did not decrease significantly during treatment with Van at either concentration used. This is not surprising because Van is less effective against non-growing cultures than against growing cultures. Rifalazil by itself had only a modest effect, decreasing the cfu by about 1 log. When combined with either concentration of Van, Rfz showed an enhanced killing effect, dropping the cfu/ml in these cultures by approximately 3 logs at 48 hours.

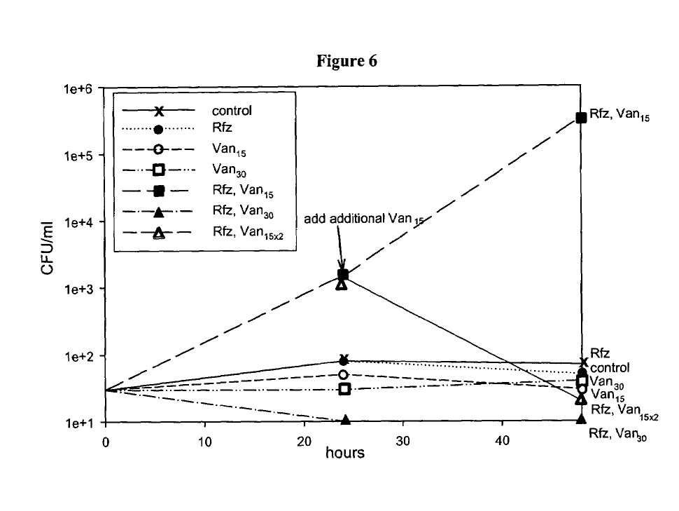

The emergence of Rif/Rfz-resistant cells in cultures in these experiments was monitored as previously described; the results are shown in FIG. 6. Resistant colonies did not appear in Van or Rfz treated cultures over the course of the experiment. For Rfz, this was in contrast to what was seen in log-phase cultures. Outgrowth of the Rfz-resistant subpopulation requires cell growth, and these cultures are not growing. Resistant mutants did arise in the culture treated with Van at 15 μg/ml plus Rfz. It is assumed that for this culture, comparatively low levels of Van plus Rfz result in some killing of the culture, and therefore nutrients are made available allowing the culture to enter “pseudo-log phase” growth. This growth allows for the emergence of some of the resistant cells over the course of the experiment. Addition of more Van or a higher level of Van at the start of the experiment presumably is effective at killing a larger proportion of these cells that have entered into a growth phase.

### Materials and Methods

The foregoing results were obtained using the following materials and methods.

### Materials

The bacterial strain used in this study was “wild-type” S. aureus strain ATCC 29213. The strain was grown in Mueller Hinton II Broth (MHB; cation-adjusted; VWR) and on Mueller Hinton Agar (MHA; VWR) plates that were made according to the manufacturer's instructions. Van (Calbiochem) was resuspended in sterile water (Fisher) at 10 mg/ml, while Rif (Calbiochem) and Rfz (ActivBiotics) were resuspended in 100% DMSO (J. T. Baker) at 1 mg/ml. All drugs were stored at −80° C. in single-use aliquots.

### Methods

S. aureus strain ATCC 29213 was grown on an MHA plate at 35° C. for 18 hours. Colonies (3-5) were used to inoculate 50-100 ml of cation-adjusted MHB in a 500 ml Ehrlenmeyer flask and grown at 37° C. in a water bath shaker at 240 rpm.

For log-phase studies, cells were grown to an optical density of 0.5 at 600 nm, which represents a culture density of 5×10⁸ colony forming units (cfu)/ml for S. aureus and deviates from densities typically used in time-kill type assays (1×10⁷ cfu/ml). We chose to start at a higher density because this density is more likely to reflect in vivo bioburden than lower starting densities. In addition, this starting density is critical for monitoring the emergence of ansamycin-resistant mutants because it insures that there is a starting resident population of such resistant mutants at the start of the experiment. The 10⁻⁸ frequency of mutants to Rfz leads to a theoretical number of five mutants per ml of culture at this cell density.

For stationary phase studies, cultures were grown to an optical density of 2.2 to 2.4 at 600 nm, which represents approximately 5×10⁹ cfu/ml; there would exist approximately 50 resistant mutants per ml in such a stationary phase culture. Drugs were added to the cultures to start the experiment, and then aliquots were removed at various time points, diluted in MHB if appropriate, and applied to 100 mm MHA and MHA plates containing Rif at 1 μg/ml. These plates were incubated at 35° C. for 18-24 hours, and then colonies manually counted to determine the number of cfu per ml of culture for the specific time point/treatment. Enumeration of Rif-resistant cells was used as a monitor of Rfz-resistant cells because we have found that cells that are Rfz-resistant are cross-resistant to Rif. The Rif-containing plates were used to determine the number of Rfz-resistant bacteria present in the cultures for the specific time-point/treatment. Untreated control cultures were included in each experiment.

### OTHER EMBODIMENTS

All patent applications and publications mentioned in this specification are herein incorporated by reference to the same extent as if each independent patent application and publication was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains. Other embodiments are within the claims.

What is claimed is:

1-26. (canceled)

27. A method for treating gastrointestinal tract infections in a patient, comprising administering to the patient a composition comprising rifalazil.

28. The method of claim 27, wherein the infection is caused by Clostridium difficile.

29. The method of claim 27, wherein the infection is caused by Bacillus anthracis.

30. The method of claim 27, wherein said administering is for a duration and in an amount that is effective to treat said patient.

31. The method of claim 27, wherein the rifalazil is administered orally.

32. The method of claim 27, wherein the dosage of rifalazil is 0.001 to 1000 mg/day.

33. A method for treating a patient suffering from an inflammatory disease associated with a gastrointestinal tract infection, comprising administering to the patient a composition comprising rifalazil.

34. The method of claim 33, wherein the infection is caused by Clostridium difficile.

35. The method of claim 33, wherein wherein the infection is caused by Bacillus anthracis.

36. The method of claim 33, wherein said administering is for a duration and in an amount that is effective to treat said patient.

37. The method of claim 33, wherein the rifalazil is administered orally.

38. The method of claim 33, wherein the dosage of rifalazil is 0.001 to 1000 mg/day.

39. The method of claim 33, wherein the inflammatory disease is selected from the group consisting of chronic inflammatory bowel disease, ulcerative colitis, and Crohn's disease.

40. The method of claim 33, wherein the inflammatory disease is chronic inflammatory bowel disease.

41. The method of claim 33, wherein the inflammatory disease is antibiotic-associated colitis.

42. The method of claim 33, wherein the inflammatory disease is Crohn's disease.

43. A method of treating pelvic inflammatory disorder associated with a bacterial infection, comprising administering rifalazil.

44. The method of claim 43, wherein the bacteria is Chlamydia trachomatis.

45. The method of claim 43, wherein said administering is for a duration and in an amount that is effective to treat said patient.

46. The method of claim 43, wherein the rifalazil is administered orally.

47. The method of claim 43, wherein the dosage of rifalazil is 0.001 to 1000 mg/day.

48. A method of treating a Chlamydia trachomatis infection in a patient in need of treatment thereof, comprising administering rifalazil to the patient.

49. The method of claim 48, wherein said administering is for a duration and in an amount that is effective to treat said patient.

50. The method of claim 48, wherein the rifalazil is administered orally.

51. The method of claim 48, wherein the dosage of rifalazil is 0.001 to 1000mg/day.

52. The method of claim 41, wherein the antibiotic-associated colitis is caused by vancomycin resistant enterococci infection.

53. A method of treating a patient suffering from a bacterial infection caused by bacteria with a multiplying and a non-multiplying phase, comprising administering a composition of rifalazil and vancomycin.

54. The method of claim 53, wherein the bacteria include vancomycin resistant enterococci spp.
